(19) United States
(12) Patent Application Publication
(10) Pub. No.: US 2007/0067887 A1
(43) Pub. Date: Mar. 29, 2007

(54) Safety hat with adjustable visor

(76) Inventor: Anthony Wang Lee, Yung Kang City (TW)

Correspondence Address: NIKOLAI & MERSEREAU, P.A., 900 SECOND AVENUE SOUTH, SUITE 820, MINNEAPOLIS, MN, 55402, US

(21) Appl. No.: 11/235,718

(22) Filed: Sep. 26, 2005

(51) Int. Cl. A61F 9/00

(52) U.S. Cl. 2/12

(57) ABSTRACT

A safety hat with an adjustable visor has a crown and a visor mounted in front of the crown. Two positioning holes are defined in two sides of the crown and multiple first teeth are formed around each positioning hole. A plurality of rods is mounted in an inner wall of the crown with a lug formed at a top of a body of each rod. Two holes are defined in the arcuate wall to receive the rods of the crown. Two buttons each with a stub are provided in the arcuate wall. By pressing the buttons, the inclination of the visor relative to the crown can be changed, and the position of crown is not changed.

### BACKGROUND OF THE INVENTION

1. Field of the Invention

The present invention relates to a safety hat, and particularly relates to a safety hat in which a tilt direction of the visor can be adjusted and without altering position of a crown of the hat.

2. Description of Related Art

Wearing safety hats is compulsory in building sites to protect the heads of personnel. The present safety hat has a crown and a visor integrally formed in front of the crown, but the visor inclination can not be changed unless the whole safety hat direction is changed. Sometimes workers have to press down the visor of the hat to prevent the strong light coming to the eye; however, the rear part of the skull and brain are exposed in that occasion, which is dangerous to the workers. In addition, the inclined safety hat is not comfortable for wearing and may cause distracting irritation to the wearer.

Therefore, the invention provides a safety hat with an adjustable visor tilt direction to mitigate or obviate the aforementioned problems.

### SUMMARY OF THE INVENTION

The main objective of the present invention is to provide a safety hat with an adjustable visor inclination that has a crown and a visor, the visor being mounted in front of the crown. The cover has a two positioning holes respectively defined in two sides of the crown, and multiple rods are mounted in an inner wall of the crown.

The visor has at least one button than can be pressed to adjust the visor tilt. The safety hat of this invention will not impair the protection of the head when the visor's inclination direction is changed and the safety hat is comfortable.

Other objectives, advantages and novel features of the invention will become more apparent from the following detailed description when taken in conjunction with the accompanying drawings.

### BRIEF DESCRIPTION OF THE DRAWINGS

FIG. 1 is an exploded view of the safety hat in accordance with this invention;

FIG. 2 is a side view of the safety hat in accordance with this invention;

FIG. 3 is a cross sectional side view of the safety hat along line 3-3 in FIG. 2 when the visor's inclination has been adjusted;

FIG. 4 is a schematic illustration of the safety hat when adjusting the visor's inclination direction; and

FIG. 5 is a schematic illustration of the safety hat when the visor's inclination direction has been adjusted.

### DETAILED DESCRIPTION OF THE PREFERRED EMBODIMENT

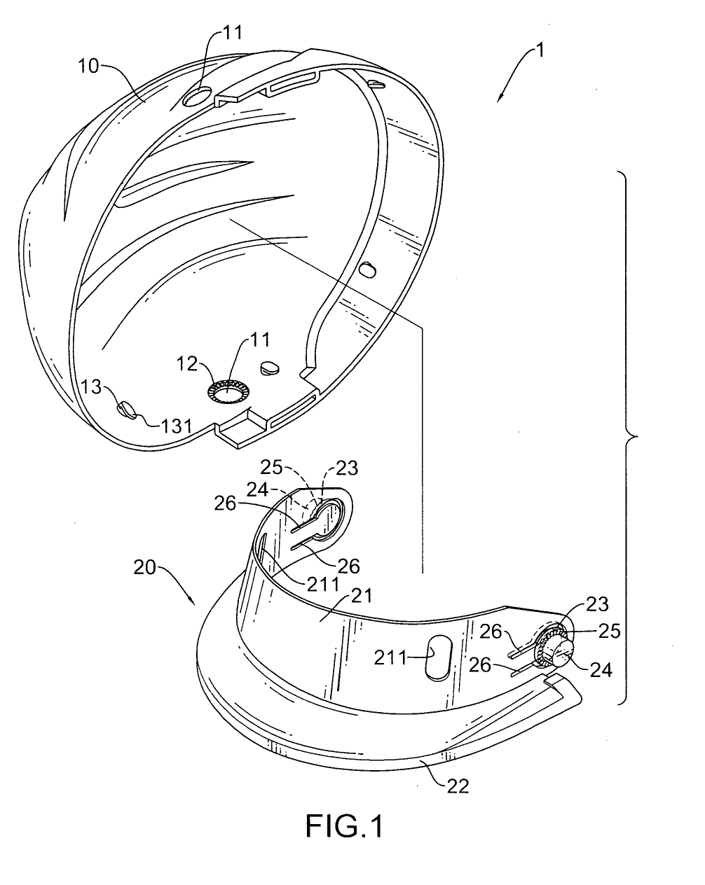

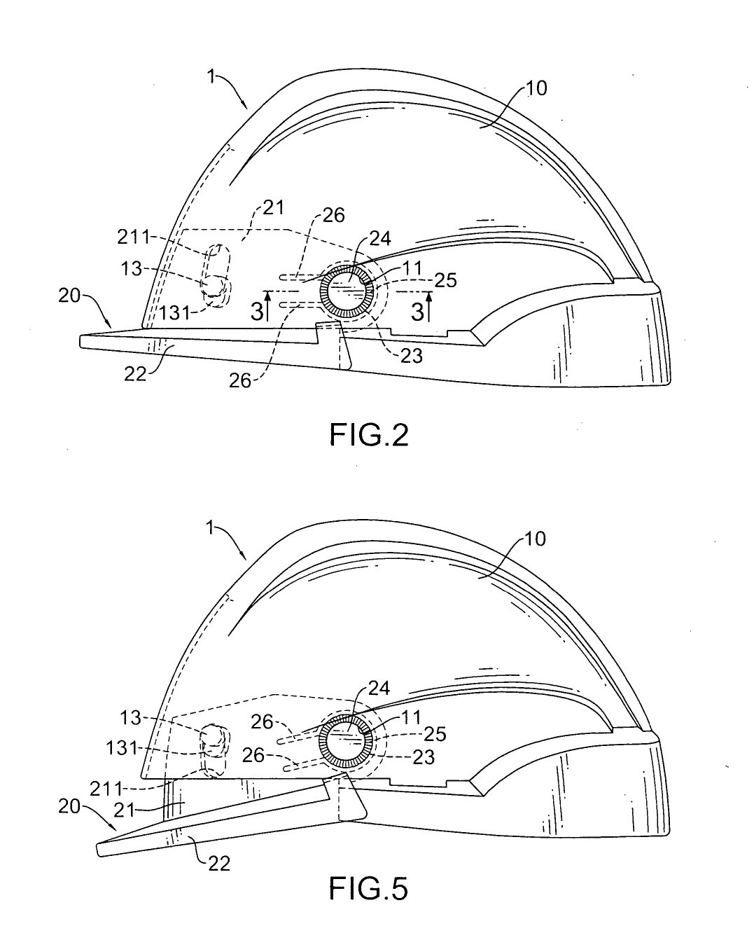

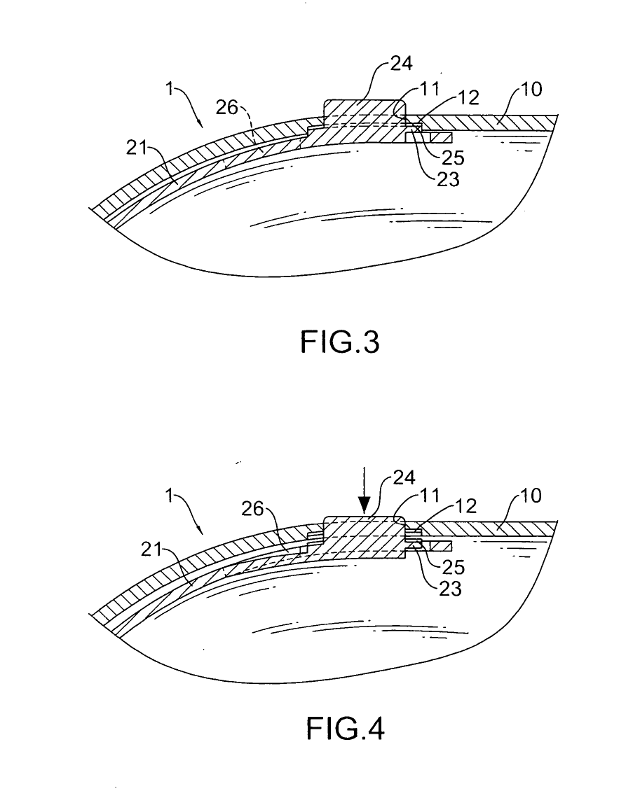

With reference to FIG. 1-3, the safety hat (1) has a crown (10), and two positioning holes (11) are respectively defined in two sides of the crown (10). Multiple first teeth (12) are formed around the periphery defining each positioning hole (11). A plurality of rods (13) is formed in an inner face of the crown (10) and each rod (13) comprises a lug (131) formed at a top of a body of the rod (13).

A visor (20) mounted in front of the crown (10) includes an arcuate inclined wall (21) and a tongue (22) extended from a bottom of the arcuate wall (21), and two holes (211) defined in the arcuate wall (21) to respectively receive two corresponding rods (13) of the crown (10). Two resilient buttons (23) are respectively provided near ends of the arcuate wall (21), and each button (23) includes a stub (24) outwardly mounted thereon. Multiple second teeth (25) are formed around the periphery of each stub (24), and the second teeth (25) are engaged with the first teeth (12) in a normal status.

A truncated keyhole-shaped slot (26) is defined around each button (23) such that the button (23) is formed on a resilient tab. With reference to FIG. 4 and FIG. 5, when it is necessary to adjust the tilt of the visor (20), the user presses the stubs (24) inward to an interior of the crown and the second multiple teeth (25) escape from the first multiple teeth (12), thereafter the two rods (13) received in the holes (211) can move along edges of the hole (211) and inclination of the visor (20) of the safety hat (1) can be adjusted. Once the visor (20) is at the desired inclination, the user releases pressure on the stubs (24) such that the buttons (23) spring back to their previous positions of engagement among the first and second teeth (12, 25). That is, the crown (10) itself will not be affected by the movement of the visor (20), so the protecting area will not be changed due to the movement of the visor (20). Therefore, the safety hat of this invention not only protects the head but also effectively screens strong light, without employing threaded fasteners, etc.

From FIG. 5, it can be seen that the rods (13) and thus the visor (20) can move within a limited range due to the holes (211). This helps prevent the visor (20) from falling should damage occur to the buttons (24).

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A safety hat with an adjustable visor, comprising:
a crown (10) having two positioning holes (11) respectively defined in two sides of the crown, and multiple first teeth (12) formed around a periphery defining each positioning hole (11), a plurality of rods (13) formed in an inner face of the crown (10) and each rod (13) having a lug (131) in a top of a body of the rod (13);
a visor (20) pivotally mounted in front of the crown (10) and including an arcuate wall (21) and a tongue (22) extended from a bottom of the arcuate wall (21), two holes (211) defined in the arcuate wall (21) to respectively receive two corresponding rods (13) of the crown (10), and two buttons (23) provided in the arcuate wall (21), each button having a stub (24) mounted thereon, multiple second teeth (25) formed around the stub (24), and the second teeth (25) engaged with the first teeth (12) in a normal status.

2. The safety hat with an adjustable visor, as claimed in claim 1, wherein a truncated keyhole-shaped slot (26) is defined around each button (23) such that the button (23) is formed on a resilient tab.
